(19) United States
(12) Patent Application Publication
(10) Pub. No.: US 2007/0014882 A1
(43) Pub. Date: Jan. 18, 2007

(54) Ellagic acid and anti-coagulant compound

(76) Inventor: Spencer Birnberg Feldman, Makawao, HI (US)

Correspondence Address: Spencer Feldman, 2599 A Olinda Road, Makawao, HI, 96768, US

(21) Appl. No.: 11/486,662

(22) Filed: Jul. 13, 2006

Related U.S. Application Data
(60) Provisional application No. 60/699,987, filed on Jul. 13, 2005.

(51) Int. Cl. A61K 36/42; A61K 31/366

(52) U.S. Cl. 424/758; 514/453

(57) ABSTRACT

A compound comprising blood-thinning and or anti-coagulant ingredients mixed with ellagic acid and/or ellagitannin, said compound-taken orally.

### BACKGROUND

This invention relates to a method of delivery of ellagic acid and/or ellagitannin to the body.

Ellagic acid is a phenolic compound which has been proven to be anti-carcinogenic, anti-mutagenic, and an anti-cancer initiator.

Ellagitannin is the naturally occurring form of ellagic acid and becomes ellagic acid in the body through the digestive process.

Clinical studies also show that ellagic acid reduces the occurrence of birth defects, promotes wound healing, reduces and reverses chemically induced liver fibrosis, and may be helpful in the fight against heart disease.

Ellagic acid has been shown clinically to increase the clotting of blood by the stimulation of the Hageman factor also known as factor XII.

Ellagic acid is so effective at causing blood to clot that it has been used specifically for this purpose in scientific tests where clotting and causing damage is part of the research.

This invention relates to the addition of anti-coagulants to ellagic acid and or ellagitannin so that the beneficial effects of ellagic acid and/or ellagitannin can be utilized without the concurrent risk of causing potentially harmful blood clots.

### DESCRIPTION

The present invention relates to a composition containing ellagic acid and/or ellagitannin with blood timing agents added to it. These agents can include but are not limited to pumpkin seed, gingko biloba, nattokinase and bromelain.

### SUMMARY

The present invention provides a method for taking ellagic acid and/or ellagitannin without significantly increasing the risk of a blood clot by the addition of anti-coagulants.

Preferred Method

160 mg of ellagitannin is combined with 150 mg quercetin, 150 mg of pumpkin seed extract, 150 mg bromelain, 60 mg gingko biloba and 20 mg nattokinase in a capsule taken orally.

I claim:

1. A method of administering ellagic acid and/or ellagitannin mixed with one or more anti-coagulants, given to a patient orally.

2. A method of administering ellagic acid and/or ellagitannin mixed with pumpkin seeds or pumpkin seed extracts, given to a patient orally.
